(19) United States
(12) Patent Application Publication
(10) Pub. No.: US 2017/0233320 A1
(43) Pub. Date: Aug. 17, 2017

(54) PROCESS FOR CLEANING CONTAMINATED FLUID

(71) Applicants: Evan Koslow, Dallas, TX (US); Blake Smith, Perrin, TX (US)

(72) Inventors: Evan Koslow, Dallas, TX (US); Blake Smith, Perrin, TX (US)

(21) Appl. No.: 15/404,918

(22) Filed: Jan. 12, 2017

Related U.S. Application Data
(63) Continuation-in-part of application No. PCT/US2015/065040, filed on Dec. 10, 2015.

(51) Int. Cl. C07C 41/44; C07C 29/88

(52) U.S. Cl. CPC C07C 41/44; C07C 29/88

(57) ABSTRACT

A process for cleaning contaminated fluid includes combining the contaminated fluid with an acid and water to yield a mixture in which at least some contaminants in the contaminated fluid are insoluble. The insoluble contaminants precipitate to yield precipitated contaminants. At least a portion of the precipitated contaminants are separated from the mixture. At least a portion of the acid and a portion of the water in the mixture are separated from the glycol in the mixture to yield cleaned fluid.

### CROSS-REFERENCES TO RELATED APPLICATIONS

This application is a continuation-in-part of PCT application no. PCT/US15/65040, having an international filing date of Dec. 10, 2015, which is incorporated herein by reference in its entirety.

### FIELD

The disclosure relates to contaminated fluid, such as glycol that has been fouled during use in a glycol dehydrating process. More specifically, the disclosure relates to processes for cleaning contaminated fluid via the precipitation of contaminants from the contaminated fluid.

### INTRODUCTION

Glycol dehydrating processes are widely used in natural gas processing plants, for the reduction of moisture in natural gas and preparation of this gas for pipeline distribution. Triethylene glycol (TEG) is the most common fluid used within these processes, although diethylene glycol (DEG) and other glycols are also sometimes used. In these processes, raw natural gas is passed through a bubble-tray contacting tower in a counter-current direction to a flow of TEG. This is accomplished at a relatively low temperature, where the moisture is rapidly adsorbed into the liquid.

The wet (sometimes called “rich”) TEG emerging from the process is sent to a boiler where the fluid is heated to drive off the moisture. The dry fluid (often called “lean”) is then cooled in a heat exchanger and usually filtered using a combination of a particulate filter and an activated carbon filter. This filtration aims to remove contaminants that accumulate in the glycol, such as higher hydrocarbons, particulates, and a wide variety of higher molecular weight polar contaminants (HMWPC).

One of the core problems in glycol dehydrating processes is that the current filtration technologies poorly control the accumulation of HMWPC. The accumulating HMWPC are entirely stable and fully dispersed within the glycol, and particularly in triethylene glycol due to its polar nature, and tend to pass through ordinary particulate filters. In addition, the polar nature of HMWPC discourages adsorption onto activated carbon, and even if adsorbed, they are likely to be displaced from the surface of the activated carbon by other nonpolar higher hydrocarbons.

Under these conditions, the HMWPC continue to accumulate regardless of the use of various filters, and eventually foul the entire system. The HMWPC then build up and lead to a large increase in viscosity of the fluid, are the major cause of foaming within the bubble tray tower, interfere with the absorption process, and eventually force the replacement or extensive vacuum distillation of the fluid within the system.

In addition, current filtration technologies poorly control the accumulation of low molecular weight polar contaminants (LMWPC).

There are roughly 36,000 large-scale glycol-dehydrating processing plants operating in the United States, and an even greater number throughout the world. Overall, there may be over 100,000 of these systems of various sizes operating worldwide.

### SUMMARY

The following summary is intended to introduce the reader to various aspects of the specification.

According to some aspects, a process for cleaning contaminated glycol includes obtaining a contaminated fluid that has been contaminated with poly(carboxyl) compounds, and b) combining the contaminated fluid with an acid and water to yield a mixture in which at least some contaminants in the contaminated fluid are insoluble. The insoluble contaminants precipitate to yield precipitated contaminants. The process further includes c) separating at least a portion of the precipitated contaminants from the mixture, and d) separating at least a portion of the acid and the water in the mixture from the fluid in the mixture to yield cleaned fluid.

In step b), the temperature of the mixture may be elevated. The elevated temperature may be at least about 140 degrees F., or between about 160 degrees F. and 212 degrees F., or about 190 degrees F., or at least about 190 degrees F.

Step b) may include heating at least one of the contaminated fluid, the acid, and the water to the elevated temperature, and then combining the contaminated fluid, the acid, and the water. Alternatively or additionally, step b) may include combining the contaminated fluid, the acid, and the water, and then heating the mixture to the elevated temperature.

Step c) may be carried out at most 15 minutes after the initiation of step b), or at between 5 minutes and 8 minutes after the initiation of step b).

The contaminated fluid may in some examples not be filtered prior to step b), because it has been found that under certain conditions, the presence of particulate contamination in the contaminated fluid promotes the more rapid formation of precipitates in step b) and creates precipitates that are more stable.

The contaminated fluid may be or may include at least one of triethylene glycol, diethylene glycol, tetraethylene glycol, and ethylene glycol. In some particular examples, the contaminated glycol is triethylene glycol.

The acid may be or may include at least one of acetic acid, sulphuric acid, phosphoric acid, and hydrochloric acid. In some particular examples, the acid is acetic acid.

The water to contaminated fluid ratio in the mixture may be at least about 1:2 by weight, or between about 1:2 and about 2:1 by weight, or about 1:1 by weight, or about 2:1 by weight, or at least 2:1 by weight.

The amount of acid in the mixture, as measured by weight % of the contaminated fluid in the mixture, may be at least about 0.25% or at least about 1.0%, or between about 0.25% and about 30%, or between about 0.5% and about 10%, or between about 1% and about 1.3%.

Step c) may include centrifugation, filtration, or other solid-liquid separation techniques such that a portion of the precipitated contaminants are separated from the mixture. Step c) may include filtering the mixture through a nano fiber paper, and/or a 1-micron filter paper. Step c) may include filtering the mixture through a coarse filter (e.g. in the 5-10 micron rating range), but at an initial pressure of less than 0.3 bar. Following the development of a dirt cake on the filter medium, it has been found that the filtration can continue at progressively higher differential pressure across the filter paper as the dirt cake thickens. The filtration process can be operated at a fixed flow rate that creates a low flow density (flow per unit area of filter medium) so that the extrusion of the soft precipitated contaminant through the filter paper is avoided. Once a thick dirt cake has developed, the contaminant has a reduced tendency to extrude through the filter medium. The criticality of this low flow density becomes more acute as the filter medium's pores become larger than 1 micron as determined by such standard methods of capillary porometry.

Step c) can include initially filtering the mixture through a filter paper with an average pore size of 1.0 to 2.5 microns at a pressure less than 0.5 bar by operating at a low flow rate through the filter medium or by restricting applied force across the filter medium. Alternatively, step c) can include filtering the mixture through a filter medium of 2.5 to 5.0 micron average pore size at a pressure of less than 0.3 bar by operating at a low flow rate through the filter medium or by restricting applied force across the filter medium.

Step (c) can include filtering the mixture through sub-micron filter media, and/or filtering the mixture through an adsorbent.

The process may further include after step c), e) contacting the mixture with an ion exchange resin to remove salts from the mixture. The ion exchange resin may be a cationic exchange resin, anion exchange resin, or a sequence of anion and cation exchange resins, or a mixed cationic and anionic exchange resin. In some examples, step d) may include contacting the mixture with a cationic exchange resin and then contacting the mixture with an anionic exchange resin, or contacting me mixture with an anionic exchange resin and then contacting the mixture with a cationic exchange resin.

The process may be one of a continuous process, a semi-continuous process, or a batch process.

The contaminants may be or may include high molecular weight polar contaminants. The contaminants may be or may include high molecular weight poly(carboxyl) compounds. The contaminants may be or may include low molecular weight polar contaminants. The contaminants may be or may include low molecular weight poly(carboxyl) compounds. The contaminants may be or may include one or more of polar carboxylic acids, polycarboxylic acids, fatty acids, cross-linked carboxylic acids, and polymerized polycarboxylic acids.

Step d) may include evaporating the portion of the water and acid from the glycol. Step d) may further include condensing the evaporated water and acid and recycling the condensed water and acid to step b).

The contaminated fluid may be contaminated glycol, such as glycol obtained from a glycol dehydrating process of a natural gas processing plant.

The contaminated fluid may include at least one of lube oil and glycol.

According to another aspect; a process for precipitating contaminants from contaminated fluid includes: a) combining the contaminated fluid with water and acid to yield a mixture in which at least a portion of the contaminants in the fluid are insoluble. The insoluble contaminants precipitate to yield precipitated contaminants.

The process may be carried out at an elevated temperature. The elevated temperature may be at least about 140 degrees F., or between about 160 degrees F. and 212 degrees F., or at least about 190 degrees F.

Step a) can include heating at least one of the contaminated fluid, the acid, and the water to the elevated temperature, and then combining the contaminated fluid, the acid, and the water. Alternatively or additionally, step a) can include combining the contaminated fluid, the acid, and the water, and then heating the mixture to the elevated temperature.

In some cases, the contaminated fluid may not be filtered prior to step a).

The contaminated fluid may be or may include at least one of triethylene glycol, diethylene glycol, tetraethylene glycol, and ethylene glycol. In some particular examples, the contaminated glycol may be triethylene glycol.

The acid may be or may include at least one of acetic acid, sulphuric acid, phosphoric acid, and hydrochloric acid. In some particular examples, the acid is acetic acid.

The water to fluid ratio in the mixture may be at least about 1:2 by weight, or between about 1:2 and about 2:1 by weight, or about 1:1 by weight, or about 2:1 by weight, or at least about 1:2 by weight.

The amount of acid in the mixture, as measured by weight % of the glycol in the mixture, may be at least about 0.25%, or at least about 1.0%, or between about 0.25% and about 30%, or between about 0.5% and about 10%, or between about 1% and about 1.3%.

The process may be carried out at greater than about 140 degrees F., or between about 160 degrees F. and 212 degrees F., or at about 190 degrees F., or at least about 190 degrees F.

The contaminants may be or may include high molecular weight polar contaminants. The contaminants may be or may include high molecular weight poly(carboxyl) compounds. The contaminants may be or may include low molecular weight polar contaminants. The contaminants may be or may include low molecular weight poly(carboxyl) compounds. The contaminants may include one or more of polar carboxylic acids, polycarboxylic acids, fatty acids, cross-linked carboxylic acids, and polymerized polycarboxylic acids.

The contaminated fluid may be glycol obtained from a glycol dehydrating process of a natural gas processing plant

According to some aspects, a process for cleaning contaminated triethylene glycol includes a) combining the contaminated triethylene glycol with acetic acid and water. The water to glycol ratio is between about 1:1 and about 1:2 by weight and the amount of acetic acid in the mixture, as measured by weight % of the glycol in the mixture, is between about 0.25% and about 30%. At least some high molecular weight polar contaminants in the glycol are insoluble in the mixture, and the insoluble high molecular weight polar contaminants precipitate from the mixture to yield precipitated high molecular weight polar contaminants. The process further includes b) separating at least a portion of the precipitated high molecular weight polar contaminants from the mixture, and c) separating at least a portion of the water and acetic acid in the mixture from the glycol in the mixture by, for example, evaporating a portion of the water and acetic acid.

The mixture of water, acid and glycol may be heated to greater than 140 degrees F.

Step b) may include filtering the mixture through sub-micron filter media. Step b) may include filtering the mixture through an adsorbent.

According to some aspects, acetic acid and water are used to precipitate contaminants from contaminated glycol that has been contaminated in a glycol dehydrating process of a natural gas processing plant. The acetic acid and water combine with the contaminated glycol to yield a miscible mixture in which at least a portion of the contaminants are insoluble.

According to some aspects, a process for cleaning contaminated fluid that has been contaminated with poly(carboxyl) compounds includes a) combining the contaminated fluid with a coagulant and a flocculant to coagulate and floc at least some contaminants of the contaminated fluid and yield a mixture of flocculated contaminants and a cleaned fluid; and b) separating at least a portion of the flocculated contaminants from the cleaned fluid.

The coagulant may include alum. The flocculant may include poly(DADMAC).

Step b) can include filtering the mixture through a sub-micron filter media. Step b) can include filtering the mixture through an adsorbent. Step b) can include filtering the mixture through a filter media comprising at least one of nanofibers, electrically charged nanofibers, and a finely divided absorbent.

Step b) can include filtering the mixture through a sub-micron filter media containing an absorbent for intercepting colloidal precipitate and contaminant at the molecular level.

In any of the above aspects, separation may involve filtration, and filtration may involve the use of an absorbent.

### BRIEF DESCRIPTION OF THE DRAWINGS

The drawings included herewith are for illustrating various examples of articles, methods, and apparatuses of the present specification and is not intended to limit the scope of what is taught in any way. In the drawings:

FIG. 1 is a flow diagram of an example process for cleaning contaminants from contaminated glycol;

FIG. 2 is a flow diagram of another example process for cleaning contaminants from contaminated glycol; and

FIG. 3 is a graph showing the percent reduction in contaminants in a triethylene glycol sample plotted against the amount of acetic acid (by weight of glycol) used to precipitate the contaminants.

### DETAILED DESCRIPTION

Various apparatuses or processes will be described below to provide an example of an embodiment of the claimed subject matter. No embodiment described below limits any claim and any claim may cover processes or apparatuses that differ from those described below. The claims are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any apparatus or process described below and for which an exclusive right is not granted by issuance of this patent application may be the subject matter of another protective instrument, for example, a continuing patent application; and the applicants, inventors or owners do not intend to abandon, disclaim or dedicate to the public any such apparatus or process by its disclosure in this document.

Processes are disclosed herein for cleaning contaminants from contaminated fluids, such as lube oils and/or glycol. The contaminated glycol may in some examples be contaminated glycol that has been contaminated in a glycol dehydrating process of a natural gas processing plant.

In some examples, the contaminated fluid may be cleaned by combining the contaminated fluid with an acid and water, optionally at an elevated temperature (i.e. with the application of heat). This yields a mixture in which at least a portion of the contaminants in the fluid are insoluble (i.e. fully or partially insoluble). The insoluble contaminants precipitate from the mixture, to yield precipitated contaminants in solid form. At least a portion of the precipitated contaminants may then be separated from the mixture. Furthermore, at least a portion of the acid and the water in the mixture may be separated from the fluid in the mixture, to yield cleaned fluid.

The processes described herein may be used to clean all, most, or some contaminants from the contaminated fluid. For example, all, most, or some of the contaminants in the fluid may be insoluble in the mixture and precipitate to yield precipitated contaminants. Furthermore, all, most, or some of the precipitated contaminants may be separated from the mixture. Furthermore, all most or some of the acid and water may be separated from the fluid. Accordingly, the term “cleaned fluid” or “cleaned glycol” is used herein as a relative term, to indicate that the fluid that is the end product of the process is clean relative to the contaminated fluid that is a starting product in the process. As such, the term “cleaned fluid” or “cleaned glycol” may refer to fluid that still contains some contaminants and/or some acid and/or some water.

Without being limited by theory, it is believed that the contaminants include HMWPC such as high molecular weight poly(carboxyl) compounds, and LMWPC such as low molecular weight poly(carboxyl) compounds. The contaminants can be composed of or can include a complex mixture of polar carboxylic acids, polycarboxylic acids, fatty acids, cross-linked carboxylic acids, and polymerized polycarboxylic acids. Because many of these are charged molecules and associated with counter-ions, they contribute to the accumulation of salt within the fluid.

It is believed that the precipitation of the contaminants from the contaminated fluid, particularly glycol, is accomplished by adjusting the Hansen Solubility Parameters of the solvent (e.g. glycol) and the contaminants such that they become generally incompatible. The glycol may be shifted to higher polarity, for example, through the addition of water, which is miscible with the glycol but is of higher polarity and has greater hydrogen bonding than the original glycol. The contaminants may also be shifted, for example, to a lower polarity through an alteration in pH. It is believed that as the mixture of glycol and water becomes more polar and the contaminants become less polar, this facilitates separation of the contaminants from the fluid because the two materials eventually are not compatible and cease being soluble.

Since the glycol is mixed with water, a more polar compound, the mixture becomes more polar. The contaminants are believed to contain compounds that are polar, e.g. poly(carboxyl) compounds. However, by adding acid to the mixture, it is believed that these compounds transition from a polar and ionized form to a non-polar and non-ionized form.

R—COO(−)+H+→RCOOH

It is believed that the negatively charged carboxyl groups transition to less polar form at the reduced pH provided by the acid, and this causes the contaminants to no longer be stable in the more polar mixture of water and glycol.

It is also believed that under certain conditions, when excessive amounts of acid are added to the mixture, this causes the contaminants to re-ionize to a positively charged form. For this reason, under certain conditions, there is an optimum for the amount of acid to produce a non-polar form of the contaminants.

In examples wherein the contaminated fluid is contaminated glycol, the contaminated glycol may be or may include any type of glycol, including but not limited to triethylene glycol (TEG), diethylene glycol, ethylene glycol, tetraethylene glycol, and combinations thereof. In some particular examples, the contaminated glycol is triethylene glycol. In some particular examples, the glycol is obtained from a glycol dehydrating process of a natural gas processing plant.

It is believed that a wide variety of acids may be suitable to cause precipitation of contaminants. For example, the acid may be or may include a weak acid and/or a strong acid. For further example, the acid may be or may include an organic acid and/or a mineral acid. For further example, the acid can be or can include strong acids and/or weak acids (i.e. acids that fully ionize when mixed with water and/or acids that only partially ionize in water). In some examples, the acid may be or may include one or more of acetic acid, sulphuric acid, phosphoric acid, and hydrochloric acid. In some particular examples, the acid is acetic acid. It has been determined that in some examples, the use of acetic acid may be particularly beneficial, as it yields a precipitate that can be relatively readily removed from the mixture by solids-liquids separation techniques. Furthermore, the use of acetic acid may be relatively practical, as it is relatively safe to handle, and may not present significant metallurgical or environmental issues.

The components may be combined in a variety of weight ratios. In some examples, where the contaminated fluid is contaminated glycol, the water to glycol ratio in the mixture may be at least about 1:2 by weight, or between about 1:2 and about 2:1 by weight, or about 1:1 by weight, or about 2:1 by weight, or at least 2:1 by weight (wherein “at least” refers to the water content). It has been determined that in some examples, it may be particularly beneficial to combine the water and glycol at a weight ratio of about 1:1, as this weight ratio may yield optimal removal of contaminants from the glycol while minimizing the amount of energy required to boil off excess water.

In some examples, where the contaminated fluid is contaminated glycol, the amount of acetic acid in the mixture, as measured by the weight of acetic acid as a percentage of the weight of glycol, may be at least about 0.25% or at least about 0.3% or at least about 1.0%, or between about 0.25% and about 30%, or between about 0.5% and about 10%, or between about 1% and about 1.3%. It has been determined that in some examples, it may be particularly beneficial to utilize between about 1% and about 1.3% acetic acid (as measured as a percentage of the weight of glycol), as this amount may yield optimal removal of contaminants from the glycol.

In some examples, the pH of the mixture may be between about 3.4 and 4.0.

The components (i.e. the water, the contaminated fluid, and the acid) may be combined at an elevated temperature. That is, heat may be applied either to the components of the mixture or to the mixture itself. In some examples, the components may be preheated to an elevated temperature, and then combined. Alternatively or additionally, the components may be combined and then the mixture may be heated to an elevated temperature. In some examples, the components may be combined at an elevated temperature of about 140 degrees F., or between about 160 degrees F. and about 212 degrees F., or at least about 190 degrees F. It has been determined that in some examples, it may be particularly beneficial to combine the components at an elevated temperature of about or at least about 190 degrees F., as this temperature may yield optimal removal of contaminants from the glycol.

In some examples, heat may be applied to the mixture for a period of time, such as a short period of time. In other words, after the initiation of the combination of the components, a period of time may lapse before the initiation of the separation of the components. For example, heat may be applied to the mixture for at least 2 minutes, or about 2 minutes or between about 5 minutes and about 8 minutes or at most about 15 minutes. It has been determined that a large proportion of the contaminants can precipitate after a short period of time.

In some examples, the contaminants cleaned from the fluid may be or may include HMWPC and/or LMWPC. In some examples, the contaminants cleaned from the fluid may be or may include high molecular weight poly(carboxyl) compounds, and/or low molecular weight poly(carboxyl) compounds. The contaminants can include polar carboxylic acids, polycarboxylic acids, fatty acids, cross-linked carboxylic acids, polymerized polycarboxylic acids, and combinations thereof. In other examples, other contaminants may alternatively or additionally be cleaned. In some cases, these compounds are associated with counter-ions or salts that may co-precipitate during this process.

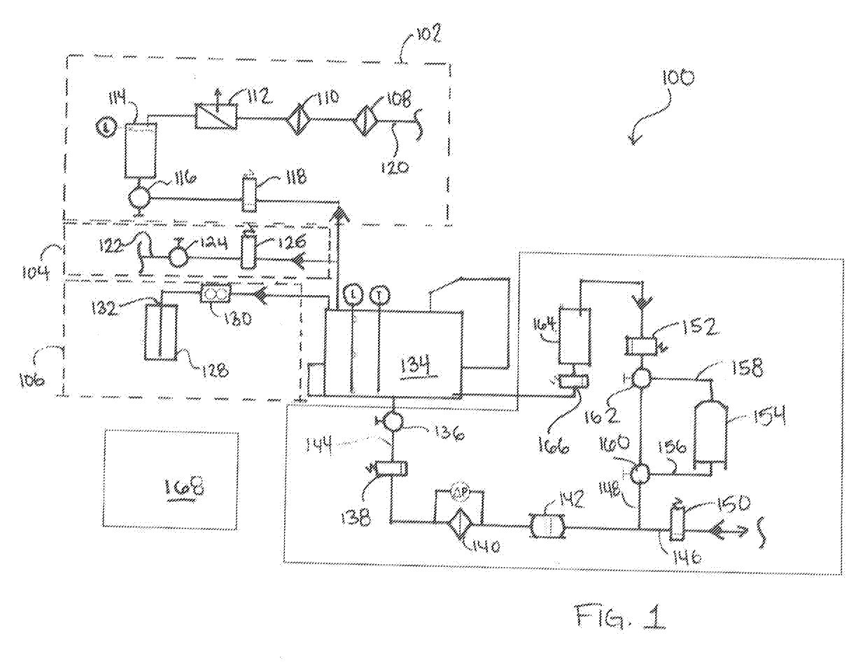

An example of a system for cleaning contaminated fluid will now be described with reference to FIG. 1. The system will be described in conjunction with an example process for operating the system. As will be described in further detail, the example process is a two-phase process for cleaning contaminated glycol. In the first phase, the contaminated glycol is combined with acid and water to precipitate at least a portion of the contaminants in the glycol, and at least a portion of the precipitated contaminants are then separated from the glycol. In the second phase, at least a portion of the acid and water are separated from the glycol.

Referring now to FIG. 1, in the example shown, the system 100 includes a water supply 102, a contaminated glycol supply 104, and an acid supply 106. The contaminated glycol may in some examples be obtained from a glycol dehydrating process of a natural gas processing plant.

The water supply may include various purification devices, such as filters and/or membranes, various storage devices such as tanks, and various valves, flowmeters, etc. In the example shown, the water supply 102 includes a polypropylene depth filter 108, a carbon filter 110, a reverse osmosis membrane 112, a surge tank 114, a shut-off valve 116, and a valve 118. Water may be fed from a source (not shown) via water line 120, through the polypropylene filter 108, the carbon filter 110, and the reverse osmosis membrane 112, and into the surge tank 114 for storage. When the system is in the first phase of operation, described in further detail below, the water is fed from the surge tank 114 past shut-off valve 116 and through the valve 118, optionally under the force of gravity, or under pressure supplied by the surge tank, which can contain a bladder (not shown) that sustains pressure within the tank.

The contaminated glycol supply 104 may include various storage devices such as tanks, and various valves, flowmeters, etc. In the example shown, the glycol supply 104 includes a glycol feed line 122, a shut-off valve 124, and a valve 126. The glycol feed line 122 may be connected directly to a source of contaminated glycol, such as a glycol dehydrating process of a natural gas processing plant (not shown). Alternatively, the glycol may be obtained indirectly from a glycol dehydrating process of a natural gas processing plant. In some examples, the contaminated glycol is not filtered prior to entering the system 100. In other examples, the contaminated glycol may be filtered. When the system is in the first phase of operation, contaminated glycol is fed from the source of contaminated glycol into line 122, past shut-off valve 124, and through valve 126, optionally under the force of gravity.

The acid supply may include various storage devices such as tanks, and various valves, flowmeters, etc. In the example shown, the acid supply 106 includes an acid storage tank 128, and a metering pump 130. When the system is in the first phase of operation, acid (e.g. acetic acid) is pumped by the metering pump 130 from the acid storage tank 128 via acid line 132

In the example shown, the water supply 102, glycol supply 104, and acid supply 106 are in communication with a mixing/heating tank 134. At the start of a batch, in the first phase of operation, valve 126 may be opened, so that contaminated glycol is fed from the glycol supply 104 into mixing/heating tank 134. Additionally, valve 118 may be opened, so that water is fed from the surge tank 114 to the mixing/heating tank 134. As mentioned above, the water may be fed to the mixing/heating tank so that the water to glycol ratio in the mixing/heating tank is at least about 1:2 by weight, or between about 1:2 and about 2:1 by weight, or about 1:1 by weight, or about 2:1 by weight.

Additionally, in the first phase of operation and at the start of a batch, the metering pump 130 may be operated to pump acid from the acid tank 128 into the mixing/heating tank 134. As mentioned above, the acid may be metered so that the amount of acid in the mixing/heating tank 134, as measured by the weight of acid as a percentage of the weight of glycol in the mixing/heating tank, may be at least about 0.25%, or at least about 0.3%, or at least about 1.0%, or between about 0.25% and about 30%, or between about 0.5% and about 10%, or between about 1% and about 1.3%.

The mixing/heating tank 134 may be supplied with water, glycol, and acid until the mixing/heating tank 134 is generally full, or until a desired amount of glycol to be cleaned is in the mixing/heating tank 134. At this point, valves 118 and 126 may be closed, and meter pump 130 may be shut off.

In the mixing/heating tank 134, the contaminated glycol is combined with the acid and the water, and the resulting mixture (also referred to herein as a “glycol/water/acid mixture”, or simply a “mixture”) is heated. As mentioned above, the mixture may be heated to at least about 140 degrees F., or between about 160 degrees F. and about 212 degrees F., or at least about 190 degrees F. The mixture may also be stirred, for example gently stirred, or otherwise agitated.

At least a portion of the contaminants in the glycol are insoluble in the mixture, and the insoluble contaminants precipitate from the mixture while in the heating/mixing tank 134. It may in some examples take about or at least 2 minutes, or between about 5 minutes and about 8 minutes, or at most about 15 minutes for a significant amount of contaminants to precipitate.

At least some of the precipitated contaminants may then be separated from the mixture, for example by filtering the precipitated contaminants from the mixture. The mixture may in some examples be filtered through a fine filter, such as a 1 micron or 2.5 micron filter, or a coarse filter, such as a 5 micron filter. The mixture may be filtered through various types of filters, such as a micro-glass filter, and/or a nano fiber filter, and/or a cellulose filter. The filter may in some examples be charged, such as positively charged. Filtration may in some examples be carried out at low differential pressure (e.g. less than 0.3 bar), for example where a coarse filter is used. Alternatively, filtration may be carried out at higher differential pressure (e.g. greater than 0.3 bar), for example where a fine filter is used. In some particular examples, filtration may be carried out at higher pressure using a fine filter, such as a 1-micron filter paper. In some examples, filtration may involve the use of an adsorbent, such as a bed of absorbent, a bonded block of adsorbent (e.g. an activated carbon block or a solid block of alternative adsorbent), a filter medium that is impregnated with or loaded with adsorbent (e.g. as described in U.S. Pat. No. 4,565,727, U.S. Pat. No. 4,904,343, U.S. Pat. No. 4,929,502, U.S. Pat. No. 5,180,630, and U.S. Pat. No. 5,192,604, each to Giglia, and U.S. Pat. No. 6,630,016 to Koslow) an adsorbent bonded to fibers using a dispersed binder, or an adsorbent layer laminated on a supporting substrate (e.g. as described in U.S. Pat. No. 5,792,513 to Koslow et al.). Suitable adsorbent media may include Fuller's earth, activated carbon, organoclays, and other high surface area materials. Filtration using adsorbents and/or smaller pore filters (e.g. submicron filters such as nanoporous filters) may allow for the separation of LMWPC in addition to HMWPC. That is, although HMWPC can in some examples be easily filtered using conventional filter papers, LMWPC can in some examples form a colloidal precipitate that is so fine that conventional filter media cannot efficiently intercept it, even when subjected to coagulation and flocculation (as described below). Filter media that include nanofibers, which may be electrically charged (e.g. positively charged using cationic coatings), and which may be supplemented by an adsorbent with highly specific affinity for the colloidal LMWPC and/or for the contaminant at the molecular level, can separate this contaminant from the mixture

It has been determined that the precipitated contaminants can be soft, gel-like, and unstable. The precipitated agglomerates can disintegrate if subjected to significant physical forces. In some examples, in order to prevent, minimize, or reduce disintegration of the agglomerates of precipitated contaminants, the glycol/water/acid mixture, with the precipitated contaminants therein, may be gently sucked through a filter under negative pressure so as to avoid passing the unstable material through the rotor of a pump.

In the example shown, in order to separate precipitated contaminants from the mixture, the system includes another shut-off valve 136, another valve 138, a filtration assembly 140, and a diaphragm pump 142, all downstream of the mixing/heating tank 134. After precipitation of the contaminants is complete (e.g. after a residence time following heating of at least 2 minutes or about 2 minutes in the mixing/heating tank, or when a desired amount of contaminants have precipitated), the valve 138 may be opened and the mixture and precipitated contaminants may be sucked by the diaphragm pump 142 into line 144, past the shut-off valve 136, and into the filtration assembly 140. In the filtration assembly 140, at least some of the agglomerates of precipitated contaminants are retained, and the mixture is pumped further downstream.

In the example shown, downstream of the diaphragm pump 142, line 144 branches into two lines: a cleaned glycol line 146, and a glycol/water/acid mixture line 148. The cleaned glycol line includes a valve 150, and the glycol/water/acid mixture line 140 also includes a valve 152. During the first phase of the process, the valve 150 is closed and the valve 152 is open, so that the mixture flows into the glycol/water/acid mixture line 148.

In the example shown, the glycol/water/acid mixture line 148 connects to an ion exchange column 154, via ion exchange inlet line 156 and shut-off valve 160, and ion exchange outlet line 158 and shut-off valve 162. Some or all of the mixture may be diverted from line 148 to the ion exchange column 154. In the ion exchange column, the mixture may contact an ion exchange resin to remove harmful or undesirable salts from the mixture. For example, at least some salts that lead to scale formation, such as calcium carbonate, may be removed from the mixture.

The ion exchange resin may be or may include a cationic exchange resin, or a mixed cationic and anionic exchange resin. Alternatively, in some examples, the ion exchange column 154 may include two stages, so that the mixture is first contacted with a cationic exchange resin and then contacted with an anionic exchange resin.

In the example shown, after passing through the ion exchange column 154, the mixture is fed back into line 148, and past valve 152. Downstream of valve 152 is a glycol/water/acid mixture surge tank 164, and another valve 166. During the first phase of operation, the valve 166 is closed, and the glycol/water/acid mixture surge tank 164 is filled with the glycol/water/acid mixture as it is pumped from the mixing/heating tank 134.

When the mixture has passed from the mixing/heating tank 134 into the glycol/water/acid mixture surge tank 164, the first phase of operation of the system 100 is complete, and the second phase may begin.

During the second phase of operation, at least a portion of the acid and the water in the mixture is separated from the glycol in the mixture, to yield cleaned glycol. In the example shown, in the second phase of operation, valve 138 is closed and valve 166 is open, so that the glycol/water/acid mixture is fed back into the mixing/heating tank 134, optionally under the force of gravity. In the mixing/heating tank 134, at least some of the water and acid of the mixture is evaporated from the glycol of the mixture. For example, in the mixing/heating tank 134, the mixture may be heated to about 212 degrees F., to evaporate water and acid. Carbonic acid in the mixture may also be released as carbon dioxide.

In some examples, the evaporated water and acid may be condensed and collected, so that it can be recycled to the first phase of the process. In alternative examples, the evaporated water and acid may be released to the atmosphere. This may be particularly practical in examples wherein acetic acid is used as the acid, since the release of small amounts of acetic acid to the atmosphere may not cause environmental concerns.

When a desired amount of water and acid has been separated from the glycol (e.g. all, most, or some of the water and acid), valves 138 and 150 may be opened, and valve 152 may be closed. The diaphragm pump 142 may be operated to suck the now cleaned glycol back through filter assembly 140, to remove any additional or remaining solids in the cleaned glycol. The cleaned glycol may then be pumped towards the cleaned glycol line, past the valve 150. Downstream of the valve 150, the cleaned glycol in the cleaned glycol line 146 may optionally be stored or reused. For example, cleaned glycol line 146 may feed to a cleaned glycol depository (not shown). In some examples the cleaned glycol depository is a part of the glycol dehydration plant.

The system 100 may in some examples include a controller 168, which may include a processor and a user interface, and which may be used to control various features of the system, such as temperature, pressure, and flow rates.

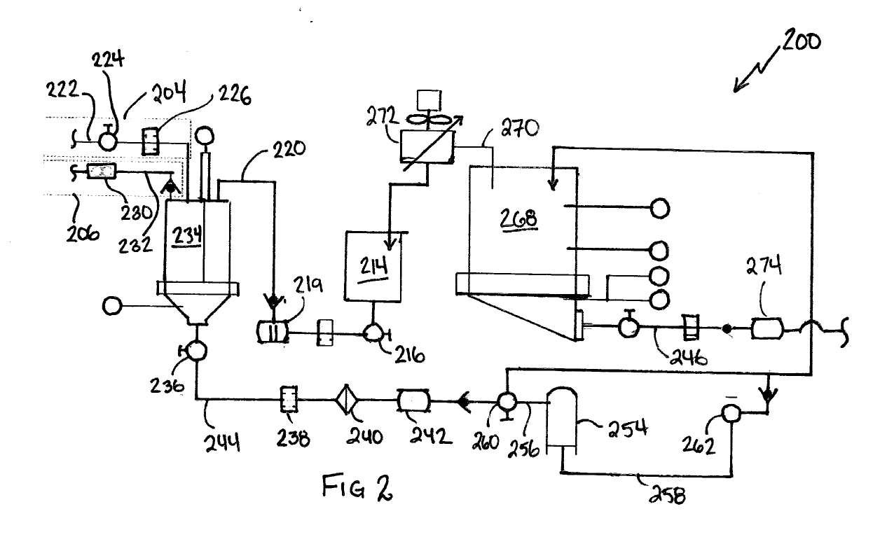

Referring now to FIG. 2, an alternative example of a system 200 for cleaning contaminated glycol is shown. The system 200 is a single phase system. Many features of system 200 are similar to features of system 100, and for simplicity, details of such features will not be repeated.

The system includes a contaminated glycol supply 204, which includes a glycol feed line 222, a shut-off valve 224, and a valve 226. The glycol feed line 222 may be connected directly or indirectly to a glycol dehydration process of a natural gas processing plant. The system 200 also includes an acid supply 206, which includes an acid storage tank (not shown), a meter pump 230, and an acid line 232. In the example shown, at steady state, water and acid are recycled through the system. Recycled water and acid are stored in condensate tank 214, which is in communication with water feed line 220 via shut-off valve 216 and pump 219. The acid supply 206 may be used to supplement the recycled acid, to account for acid that is lost in the system.

The system 200 includes a mixing/heating tank 234 (also referred to as a reactor tank). At the start of a batch, valve 226 may be opened, so that contaminated glycol is fed from the glycol supply 204 into mixing/heating tank 234. Additionally, pump 219 may be turned on, so that water is pumped from the condensate tank 214 to the mixing/heating tank 234. The water may be pumped to the mixing/heating tank so that the water to glycol ratio in the mixing/heating tank is as described above. Additionally, the meter pump 230 may be operated to pump acid from the acid tank into the mixing/heating tank 234. The acid may be metered so that the amount of acid in the mixing/heating tank 134 is as described above.

In the mixing/heating tank 234, the contaminated glycol is combined with the acid and the water, and the resulting mixture (also referred to herein as a “glycol/water/acid mixture”, or simply a “mixture”) is heated to an elevated temperature, as described above. The mixture may also be stirred or otherwise agitated. The insoluble contaminants precipitate from the mixture while in the heating/mixing tank 234. The mixture may be held and heated in the mixing/heating tank 234 for a period of time, as described above.

In the example shown, in order to separate precipitated contaminants from the mixture, the system includes another shut-off valve 236, another valve 238, a filtration assembly 240, and a diaphragm pump 242, all downstream of the mixing/heating tank 234. After precipitation of the contaminants is complete (e.g. after a set residence time in the mixing/heating tank 234, or when a desired amount of contaminants have precipitated), the valve 238 may be opened and the mixture and precipitated contaminants may be sucked by the diaphragm pump 242 into line 244, past the shut-off valve 236, and into the filtration assembly 240. In the filtration assembly 240, at least some of the agglomerates of precipitated contaminants are retained, and the mixture is pumped further downstream.

In the example shown, line 244 connects to an ion exchange column 254, via ion exchange inlet line 256 and shut-off valve 260, and ion exchange outlet line 258 and shut-off valve 262. Some or all of the mixture may be diverted from line 144 to the ion exchange column 254. In the ion exchange column 254, the mixture may contact an ion exchange resin to remove harmful or undesirable salts from the mixture.

In the example shown, after passing through the ion exchange column 254, the mixture is fed back into line 244. Line 244 connects to an evaporator tank 268. In evaporator tank 268, at least a portion of the acid and the water in the mixture is separated from the glycol in the mixture, to yield cleaned glycol. Particularly, the mixture may be heated in the evaporator tank to about 212 degrees F., to evaporate water and acid. The evaporated water and acid are fed via line 270 to condenser 272, where they are condensed to liquid form. The condensed water and acid are is fed via line 274 to the condensate tank 214, where it may be held until the beginning of the next batch.

The cleaned glycol in the evaporator tank 268 may then be pumped out of the evaporator tank 268 through cleaned glycol line 246 by diaphragm pump 274. Downstream of diaphragm pump 274, the cleaned glycol may optionally be stored or reused.

Some or all of the various valves described above may be solenoid valves.

In another example (not shown), instead of acid and water (or in addition to acid and water), one or more coagulants may be used to clean contaminated glycol. Coagulants may optionally be used together with one or more flocculants. One example of a suitable coagulant is alum. Examples of suitable flocculants may include diallyldimethylammonium chloride (DADMAC), and ampholytic terpolymers such as Merquat™.

In addition to use in cleaning glycol, coagulants as described herein (optionally together with flocculants) may be used to clean amine solutions, automotive part cleaning solutions (which typically include Naphtha), used motor oil, and other products contaminated with poly(anionic) contaminants such as poly(carboxyl) compounds.

Examples involving the use of a coagulant may be carried out at room temperature, or below room temperature (e.g. at or around 7 degrees Celsius), and can produce results over short time spans (e.g. within a few minutes). However, the use of higher temperatures may also be possible.

The use of a coagulant may result in the formation of a fine, nearly colloidal precipitate that remains in suspension and does not produce a heavy thick sludge, and may thus be less likely to foul equipment, cause corrosion, or have side reactions with glycol to produce methyl esters.

The example processes described above are batch processes. However, in alternative examples, alternative processes may be a continuous or semi-continuous process.

### EXAMPLES

All water used in the experiments was distilled, except during hardness and alkalinity testing, as indicated below.

All acetic acid was glacial acetic acid (A.R. grade) used at full strength unless otherwise indicated.

All results for reduction in contaminants were measured by comparing optical adsorption of the cleaned glycol product to optical adsorption of the test sample, prior to cleaning. All results reported herein are corrected for dilution by water and other ingredients.

### Series I

A sample of contaminated triethylene glycol (TEG) was obtained from a natural gas processing plant. The sample was pre-filtered through a Whatman #1 filter paper (11 micron rating). The sample is too contaminated (dark black color) to directly obtain an optical clarity reading. Therefore, a serial dilution with clean analytical grade TEG was carried out until a measure of optical clarity could be obtained. The optical clarity of the diluted sample as measured in adsorption units (AU) was multiplied by the dilution ratio to obtain a final estimated optical density of the original sample of 28.37 adsorption units (AU), as measured by a spectrometer at a wavelength of 640 nm in a standard 1 inch path length cell. This pre-filtered TEG, obtained from a natural gas processing plant, is referred to herein as the “A test sample”.

### Experiment I-1

The A test sample (10 g) was combined with distilled water (20 g) and acetic acid (2.00 g) and heated on a hot plate at 160 degrees F. for 2 minutes. This correlates to a water to glycol weight ratio of 2:1, and an acetic acid content of 20% (as measured by weight of acetic acid by weight of glycol). The resulting mixture was then filtered through a Whatman #5 filter (2.5 micron rating). Optical adsorption of the filtered mixture was measured as above to determine the reduction in contamination. It was determined that an 87.8% reduction of contamination was achieved.

### Experiment I-2—Reduced Water Content

The A test sample (10 g) was combined with distilled water (10 g) and acetic acid (2.00 g) and heated on a hot plate to boiling (212 degrees F.). This correlates to a water to glycol weight ratio of 1:1, and an acetic acid content of 20% (as measured by weight of acetic acid by weight of glycol). The resulting mixture was then filtered through a Whatman #5 filter (2.5 micron rating). Optical adsorption of the filtered mixture was measured as above to determine the reduction in contamination. It was determined that an 82.4% reduction of contamination was achieved. This result indicates that reduced water content still yields a reduction in contamination, but can reduce process efficacy under certain conditions.

### Experiment I-3—Acid Content

Experiment I-1 was repeated with varying amounts of acetic acid, and at a temperature of 140 degrees F. to 150 degrees F. Results are shown in Table 1.

TABLE 1

| | Acetic Acid | Acetic Acid | % Reduction |
|---|---|---|---|
| Sample | (absolute | (wt % of | in |
| No. | weight) | glycol) | Contaminants |
| 1 | 1.00 g | 10% | 79.5% |
| 2 | 2.17 g | 21.7% | 74% |
| 3 | 3.17 g | 31.7% | 81.1% |
| 4 | 0.00 g | 0% | <50% |

This result indicates that under certain conditions, with increasing amounts of acetic acid (i.e. up to 31.7% by weight of glycol), a further reduction in contaminants can be achieved. However, lower amounts of acetic acid still achieve modest results. This result also indicates that a modest reduction in contaminants can be achieved at a lower temperature (140 degrees F. to 150 degrees F.).

### Example I-4—Higher Temperature

Experiment I-1 was repeated with varying amounts of acetic acid, and at a temperature of 190 degrees F. Results are shown in Table 2.

TABLE 2

| | Acetic Acid | Acetic Acid: | % Reduction |
|---|---|---|---|
| Sample | (absolute | Glycol by | in |
| No. | weight) | weight | Contaminants |
| 1 | 1.00 g | 10% | 94.3% |
| 2 | 2.00 g | 20% | 84.5% |
| 3 | 3.00 g | 30% | 72.8% |
| 4 | 0.00 g | 0% | <10% |

This result indicates that increased temperature (i.e. up to 190 degrees F.) yields improved reduction in contaminants under certain conditions. This result further indicates that under certain conditions, at higher temperatures, increased amounts of acetic acid (i.e. up 30% by weight of glycol) hinder the reduction of contaminants, although a modest reduction is still achieved. This result also confirms that an absence of acid gives a nearly null result.

### Example I-5—Reduced Amount of Acetic Acid

Experiment I-4 was repeated, with varying reduced amounts of acetic acid. Results are shown in Table 3.

TABLE 3

| | Acetic Acid | Acetic Acid: | % Reduction |
|---|---|---|---|
| Sample | (absolute | Glycol by | in |
| No. | weight) | weight | Contaminants |
| 1 | 0.33 g | 3.3% | Error - sample lost |
| 2 | 0.66 g | 6.6% | 93.4% |
| 3 | 1.00 g | 10% | 95.2% |

This result indicates that under certain conditions, a decrease in the amount of acetic acid to as low as 6.6% (by weight of glycol) yields an improved reduction of contaminants. A modest reduction is still achieved at higher amounts of acetic acid (i.e. up to 10%).

### Experiment I-6—Further Reduced Amount of Acid

Experiment I-5 was repeated, with further reduced amounts of acetic acid. Results are shown in Table 4.

TABLE 4

| | Acetic Acid | Acetic Acid: | % Reduction |
|---|---|---|---|
| Sample | (absolute | Glycol by | in |
| No. | weight) | weight | Contaminants |
| 1 | 0.20 g | 2.0% | 94.1% |
| 2 | 0.40 g | 4.0% | 96.3% |
| 3 | 0.80 g | 6.0% | 97.2% |

This result indicates that under certain conditions, a further decrease in the amount of acetic acid, to as low as 2.0% (by weight of glycol) yields a significant reduction of contaminants.

### Experiment I-7—Further Reduced Amount of Acid

Experiment I-6 was repeated, with further reduced amounts of acetic acid. Results are shown in Table 5.

TABLE 5

| | Acetic Acid | Acetic Acid:Glycol | % Reduction |
|---|---|---|---|
| | (absolute | by | in |
| Sample No. | weight) | weight | Contaminants |
| 1 | 0.030 g | 0.3% | 89.3% |
| 2 | 0.050 g | 0.5% | 91.7% |
| 3 | 0.100 g | 1.0% | 96.2% |
| 4 | 0.200 g | 2.0% | 93.7% |

This result indicates that under certain conditions, an amount of acid below 1.0% (by weight of glycol) hinders the reduction in contaminants, although still achieves modest results. The optimal amount of acid (i.e. amount of acid that gave the best reduction in contaminants) appears to be about 1.0%, under certain conditions. The performance of the process with respect to the amount of acid has a broad peak, and declines to essentially zero performance in the absence of acid, and declines slowly in performance above 1% acid.

### Experiment I-8—Varying Reduced Amounts of Acid

Experiment I-7 was repeated, with varying reduced amounts of acetic acid. Results are shown in Table 6.

TABLE 6

| | Acetic Acid | Acetic Acid:Glycol | % Reduction |
|---|---|---|---|
| | (absolute | by | in |
| Sample No. | weight) | weight | Contaminants |
| 1 | 0.025 g | 0.25% | 90.7% |
| 2 | 0.050 g | 0.5% | 94.8% |
| 3 | 0.130 g | 1.3% | 97.3% |
| 4 | 0.00 g | 0.0% | 5.1% |

This result confirms that the optimal amount of acid is about 1.0%, or between about 1.0% and 1.3%, particularly at a temperature of 190 degrees F. and where the water to glycol ratio is about 2:1.

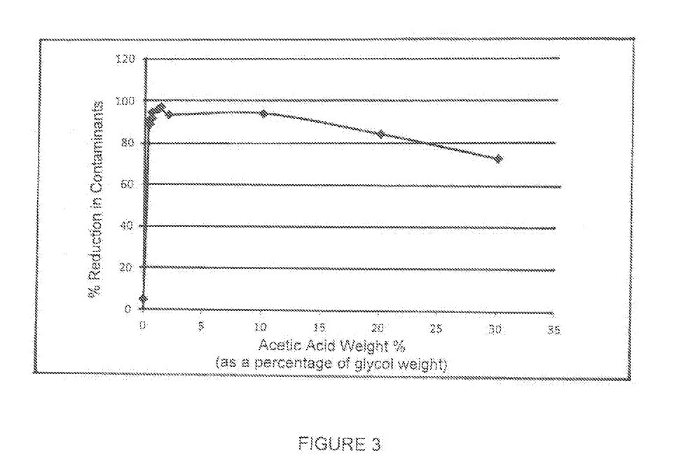

FIG. 3 of the application shows the percent reduction in contaminants plotted against the amount of acetic acid (by weight of glycol).

### Experiment I-9—Reduced Water Content

Experiment I-4 was repeated, but with varying reduced amounts of distilled water, and an amount of acetic acid constant at 1.3%. Results are shown in Table 7.

TABLE 7

| | Distilled Water | | % Reduction |
|---|---|---|---|
| | (absolute | Water:Glycol | in |
| Sample No. | weight) | by weight | Contaminants |
| 1 | 5.00 g | 1:2 | <10% |
| | | | (not readable) |
| 2 | 10.0 g | 1:1 | 38.4% |
| 3 | 15.0 g | 3:2 | 71.1% |
| 4 | 20.0 g | 2:1 | 91.6% |

This result indicates that under certain conditions, a water to glycol ratio of about 2:1 yields optimal results. Less water, down to a ratio of 2:3, yields modest results. Further decreased water still gives a reduction in contaminants, but yields very low performance. It is expected that the use of more water, i.e. a ratio of more than 2:1, would give further increased performance. However, this may require a large amount of energy, in order to boil off or otherwise remove the water.

### Experiment I-10—Phosphoric Acid

Experiment I4 was repeated, but with 0.130 g concentrated phosphoric acid (analytical grade H₃PO₄, 85% by weight) instead of acetic acid. A percent reduction in contaminants of 90.6% was achieved. This indicates that alternative acids operate effectively. However, certain acids may be less desirable due to difficulty in removal from the system. Furthermore, without being limited by theory, the efficacy of phosphoric acid indicates that the mechanism of action of the acid is not as a cross linker. Instead, the mechanism of action is believed to be in shifting the electric charge of the contaminant molecule, which causes precipitation.

### Experiment I-11—Hydrochloric Acid

Experiment I-4 was repeated, but with 0.400 g concentrated hydrochloric acid (analytical grade HCl, 37% in water). A 96.8% reduction in contaminants was achieved. This indicates that strong acids can give similar results to weak acids. It is also noted that since HCl is volatile, removal may be practical. However, for reasons of safety and metallurgy, other acids may in some examples be more practical.

### Experiment I-12—Hydrochloric Acid with Varying Amounts of Water

Experiment I-11 was repeated with varying amounts of water. The experiment was carried out at 190 degrees F. and with 0.400 g of hydrochloric acid. Results are shown in Table 8.

TABLE 8

| | Distilled Water | | % Reduction |
|---|---|---|---|
| | (absolute | Water:Glycol | in |
| Sample No. | weight) | by weight | Contaminants |
| 1 | 5.00 g | 1:2 | 48.6% |
| 2 | 10.0 g | 1:1 | 76.4% |
| 3 | 15.0 g | 3:2 | 85.7% |
| 4 | 20.0 g | 2:1 | 88.5% |

This result indicates that under certain conditions, a water to glycol ratio of about 2:1 is optimal for other acids in addition to acetic acid.

### Experiment I-13—Scalability and Filter Media

Scalability was examined by combining 200 g of A test sample with 400 g distilled water and 2.0 g acetic acid. The mixture was heated to 190 degrees F. in a 1000 mL beaker, and then filtered with a variety of media.

It was observed that if differential pressure was held at <0.2 bar (3 psid), then a 5-micron micro-glass filter medium provided a reduction in contaminants of 97.4%. When pressure was raised to 0.6 bar (9 psid), the precipitate immediately passed through the filter to produce a filtrate whose optical density was unreadable.

It was further observed that when fine filter papers (e.g. a 1-micron micro-glass filter, a 2.5-micron Whatman #5 filter, or specialty nanofiber paper (1-micron with positive charge)) were used, the precipitate did not penetrate these filters, even at elevated pressure. It was also observed that dirt holding capacity of these papers was limited. The exception was the nanofiber paper, which continued to flow while giving 96.1% reduction in contaminants. This nanofiber paper outperformed conventional filter media for dirt holding by 2-3 fold.

It was observed that a conventional pleated 5-micron micro-glass commercial filter (6.5″ O.D.×2.75″ I.D.×27″ Length) having 40 square feet of filter paper was best operated at less than 10 GPM, in order to avoid particle extrusion through the pores. As a dirt cake developed on the surface of this filter, the extrusion of material through the pores of the filter appeared to be inhibited by the presence of the previously deposited contaminant.

It was observed that dirt holding capacity rises when a filter is over-sized and flow density through the filter medium is extremely low. It is expected that the commercial filter described above (i.e. pleated 5-micro micro-glass) will operate at only 5 GPM, and thereby obtain a dirt holding capacity that allows economical change-out frequency. During the initial operation of the cleaning process at a new location, accumulated contaminant might force rapid filter change out until plant contamination levels are reduced to a more modest level.

### Series II

Another sample of contaminated triethylene glycol (TEG) was obtained from the same natural gas processing plant as in Series I.

A first sub-sample was pre-filtered through a Whatman #5 filter paper (2.5 micron rating) and diluted with clean analytical grade TEG to obtain a contamination reading of 23.00 adsorption units (AU), as measured by optical spectroscopy at a wavelength of 640 nm in a standard 1 inch path length cell. This pre-filtered TEG is referred to herein as “the B test sample”.

A second sub-sample was not pre-filtered. This non-filtered TEG is referred to herein as “the C test sample”. All filtration was carried out with 1-micron glass fiber filter medium unless specifically noted.

### Experiment II-1

Distilled water (5 g) was mixed with the B test sample (10 g) and with acetic acid (0.100 g) in a 50 glass beaker. This corresponds to a water to glycol weight ratio of 1:2, and an acetic acid content of 1.0% (as measured by weight of acetic acid by weight of glycol). The mixture was heated rapidly to 220 degrees F. (boiling) and then maintained at an elevated temperature of about 195 degrees F. for 5 minutes. The mixture was then filtered through Whatman #5 paper. It was found that the filtrate had an optical absorption of greater than 3.5 AU (not readable), and remained heavily contaminated. It should be noted that the spectrometer in use herein has a limit of 3.5 AU beyond which the optical absorption (darkness) of the sample is too great to be readable. However, in some cases, this can be defeated by dilution with clean analytical grade TEG. In such cases, when a suitable dilution has been made, the optical density can return to the effective range of the instrument and the final reading is corrected by multiplying by the dilution used to create the sample.

### Experiment II-2

Experiment II-1 was repeated with varying amounts of acid, and with the temperature of the samples brought to and not exceeding 190 degrees F. Results are shown in Table 9.

TABLE 9

| | Acetic Acid | Acetic Acid | % Reduction |
|---|---|---|---|
| | (absolute | (wt % of | in |
| Sample No. | weight) | glycol) | Contaminants |
| 1 | 0.033 g | 0.33% | Not readable |
| 2 | 0.084 g | 0.84% | Not readable |
| 3 | 0.135 g | 1.35% | Not readable |
| 4 | 0.212 g | 2.12% | >10.1 AU |

This indicates that the use of a water to glycol ratio of 1:2 and modest amounts of acid will reduce contaminants by less than 50%, under certain conditions.

### Experiment II-3

Experiment II-2 was repeated, but with higher amounts of acid. Results are shown in Table 10,

TABLE 10

| | Acetic Acid | Acetic Acid | % Reduction |
|---|---|---|---|
| | (absolute | (wt % of | in |
| Sample No. | weight) | glycol) | Contaminants |
| 1 | 0.216 g | 2.16% | Not readable |
| 2 | 0.458 g | 4.58% | Not readable |
| 3 | 0.651 g | 6.51% | Not readable |
| 4 | 0.950 g | 9.50% | 80.9% |

This result indicates that under certain conditions, the use of a water to glycol ratio of 1:2 and relatively high concentrations of acetic acid will reduce contamination by up to about 81%.

This result also indicates that relatively high amounts of acid may not compensate for reductions in the amount of water.

Samples 1 to 3 were also returned to the heater to stimulate further precipitation, but with negative results.

### Experiment II-4

Distilled water (10 g) was mixed with the B test sample (10 g), and with varying amounts of acid as shown in Table 11. This corresponds to a water to glycol ratio of 1:1. Samples were brought to a temperature of 190 degrees F. The mixture was then filtered. Results are shown in Table 11.

TABLE 11

| | Acetic Acid | Acetic Acid | % Reduction |
|---|---|---|---|
| | (absolute | (wt % of | in |
| Sample No. | weight) | glycol) | Contaminants |
| 1 | 0.030 | 0.30% | Not readable |
| 2 | 0.061 | 0.61% | Not readable |
| 3 | 0.104 | 1.04% | Not readable |
| 4 | 0.160 | 1.60% | 71.7% |

This result indicates that under certain conditions, a water to glycol ratio of 1:1 with modest acid concentrations can reduce contamination by up to about 72%.

### Experiment II-5

Experiment II-4 was repeated, but with higher amounts of acid. Results are shown in table 12.

TABLE 12

| | Acetic Acid | Acetic Acid | % Reduction |
|---|---|---|---|
| | (absolute | (wt % of | in |
| Sample No. | weight) | glycol) | Contaminants |
| 1 | 0.146 | 1.46% | Not readable |
| 2 | 0.256 | 2.56% | Not readable |
| 3 | 0.358 | 3.58% | 79.6% |

The pH of sample 3 was above 3.4.

This result indicates that under certain conditions, a water to glycol ratio of 1:1 with relatively high acid concentrations can reduce contamination by up to about 80%.

### Experiment II-6

The C test sample was used in this experiment. It is believed that this sample may mimic a commercial process, in which the contaminated glycol may not be pre-filtered. It was theorized that particulate contamination in the unfiltered sample would provide nucleation points for the precipitate.

Distilled water (10 g) was mixed with C test sample (10 g) and acetic acid (0.100 g) The mixture was brought to 190 degrees F. in approximately 4 minutes, and then filtered through a 1 micron micro-glass filter medium to obtain a measured filtrate optical density of 0.649 AU when filtered at low pressure (0.1 bar). This corresponds to a 94.5% reduction in contaminant. This improved result is believed to be due to the use of unfiltered glycol.

### Experiment II-7

In view of the results of experiment II-6, a water to glycol ratio of 1:2 was again tested using the B test sample. Distilled water (5 g) was mixed with B test sample (10 g) and acetic acid (0.109 g). This corresponds to a water to glycol ratio of 1:2, and an acetic acid content of 1.09%. The mixture was heated to 190 degrees F. The optical density was not readable

### Experiment II-8

Experiment II-7 was repeated, but with the C test sample, and with 0.117 g acetic acid. The mixture was heated to 190 degrees F. An 81.4% reduction in contamination was obtained.

This indicates that under certain conditions, the use of an unfiltered sample gives improved results. However, at low water concentrations, the reduction in contaminants was not high.

### Experiment II-9

Experiment II-8 was repeated, with varying amounts of acid. Results are shown in Table 13.

TABLE 13

| | Acetic Acid | Acetic Acid | % Reduction |
|---|---|---|---|
| | (absolute | (wt % of | in |
| Sample No. | weight) | glycol) | Contaminants |
| 1 | 0.055 | 0.55% | 79.2% |
| 2 | 0.246 | 2.46% | 64.5% |

This result indicates that under certain conditions, regardless of the amount of acid used, a water to glycol ratio of 1:2 may produce a reduction in contaminants of up to 81%, when using unfiltered glycol. The use of unfiltered glycol can provide a higher reduction in contaminants, as compared to filtered glycol.

### Experiment II-10

Distilled water (10 g) was mixed with the C test sample (10 g) with varying amounts of acetic acid, as shown in table 14. This corresponds to a water to glycol ratio of 1:1. The mixture was heated to 190 degrees F. The mixture was then filtered. Results are shown in table 14.

TABLE 14

| | Acetic Acid | Acetic Acid | % Reduction |
|---|---|---|---|
| | (absolute | (wt % of | in |
| Sample No. | weight) | glycol) | Contaminants |
| 1 | 0.028 | 0.28% | 93.6% |
| 2 | 0.071 | 0.71% | 94.6% |
| 3 | 0.147 | 1.47% | 92.9% |
| 4 | 0.200 | 2.00% | 94.8% |

These results indicate that when using a water to glycol ratio of 1:1 and a relatively low concentration of acid, superior results can be obtained using unfiltered glycol. There is a broad range of acid concentrations where greater than 93% contaminant reduction is achieved.

### Experiment II-11

A larger volume of fluid was processed in a slightly larger beaker, in order to slow the rate of heating. Water (30 g) was mixed with the B test sample (30 g) and acetic acid (approximately 0.3 g). This corresponds to a glycol to water ratio of 1:1, and an acetic acid content of 1.0%. The temperature of the mixture was raised to 190 degrees F. Samples were taken at various temperatures as the mixture was allowed to cool, and were filtered. Results are shown in table 15.

TABLE 15

| | Sample | % Reduction |
|---|---|---|
| | Temperature | in |
| Sample No. | (degrees F.) | Contaminants |
| 1 | 105 | 94.8% |
| 2 | 140 | 94.4% |
| 3 | 190 | 93.5% |

This result indicates that under certain conditions, allowing the sample to cool after reaching 190 degrees F. does not adversely impact results, if the sample is not agitated.

### Experiment II-12

The kinetics of precipitate formation were investigated. Two fluids were prepared. The first fluid was a mixture of distilled water (103 g) and acetic acid (0.81 g). The second fluid consisted of C test sample (100 g). Both fluids were heated to a temperature of 190 degrees F., and then were combined. Samples of the combined fluid were obtained at 20 second intervals and immediately filtered through a 2.5 micron-rated Whatman filter paper. Some samples were lost due to insufficient time to filter a large enough volume for optical assay. The samples shown in table 16 were successfully obtained.

TABLE 16

| | | % Reduction |
|---|---|---|
| | Sample Time | in |
| Sample No. | (seconds) | Contaminants |
| 1 | 0 | 84.6% |
| 2 | 20 | 85% |
| 3 | 120 | 90.4% |

This indicates that under certain conditions, precipitation initially occurs rapidly, and then proceeds more slowly to a final outcome. This may indicate that the reaction is governed by a second order reaction rate. This also indicates that the reaction may be incomplete within the first 2 minutes of processing, and reaches the desired contaminant reduction of 93% to 96% after more than 2 minutes.

### Experiment II-13

The impact of slowly raising the temperature of the fluid mixture was investigated. Distilled water (103 g) was combined with the C test sample (100 g) and acetic acid (0.87 g) in a 400 mL beaker. This corresponds to a water to glycol ratio of approximately 1:1 and an acetic acid content of 0.87%. The mixture was slowly heated and samples were obtained at increasing temperatures over a roughly 20 minute period. Samples were then filtered. Results are shown in Table 17.

TABLE 17

| | Sample | % Reduction |
|---|---|---|
| | Temperature | in |
| Sample No. | (degrees F.) | Contaminants |
| 1 | 125 | 95.7% |
| 2 | 144 | 93.9% |
| 3 | 175 | 93.6% |
| 4 | 185 | 90.3% |
| 5 | Boiling | 92.6% |
| | (>220 F.) | |

The percent reduction in contaminants declined continuously with temperature. This may be the result of the slow loss of water from the sample due to evaporation during the extended heating period. Upon boiling, there seems to be a partial recovery of performance.

### Experiments II-14 and II-15

The use of lower temperatures as a means to enhance performance of low water mixtures was investigated. Trials using a water to glycol ratio of 1:2 and 0.75:1 were unsuccessful (not readable). One sample having a water to glycol ratio of 1:1 and an acid content of 1% was heated to 125 degrees F. Another sample having a water to glycol ratio of 1:1 and an acid content of 1% was heated to 145 degrees F. Another sample having a water to glycol ratio of 1:1 and an acid content of 1% was left at room temperature. The two heated samples were rapidly heated, and were unreadable. The sample left at room temperature was left for one hour, and was then measured for optical density. A percent reduction in contaminants of 93% was obtained.

This experiment was repeated, using a sample having a water to glycol ratio of 1:1 and acid content of 1%, but with heating the sample to 125 degrees F. slowly. This gave a percent reduction in contaminants of 95.4%.

This experiment was repeated again, using a sample having a water to glycol ratio of 0.75:1, acid content of 1%, and with heating the sample to 125 degrees F. slowly. This gave a percent reduction in contaminants of 95.4%. This result appears to be different from prior results, and it is hypothesized that the precipitate is being “extruded” through the filter paper under certain experimental conditions.

### Experiment II-16

A mixture was prepared using water (20 g) and the C test sample (40 g) and acetic acid (0.345 g). This corresponds to a water to glycol ratio of 1:2, and an acetic acid content of 0.86%. The mixture was prepared in a 100 mL beaker, and slowly raised to 140 degrees F. An initial sample was taken after 14 minutes and was filtered. The sample was unreadable. A second sample was taken and filtered through the same filter as the first, which had a fully developed dirt cake. The second sample had a percent reduction in contaminants of 92.8%. This appears to confirm that the filtration process may be problematic under certain conditions.

It is theorized that the low temperature precipitated material is extremely soft, and even slight amounts of pressure cause the precipitate to pass through the filter. It is believed that once a dirt cake is formed, the filter can retain the soft material. This may explain the variable outcome of low-temperature processing (i.e. the precipitate forms, but filtration is variable). It is also believed that in some cases, the filter can retain the precipitate, where the pressure drop across the filter is exceptionally low—i.e. less than 1 psid.

### Experiment II-17

The C test sample (40 g) was combined with water (30 g) and acetic acid (0.311 g). This corresponds to a water to glycol ratio of 0.75:1, and an acid content of 0.78%. The temperature of the mixture was slowly raised to 140 degrees F. A sample of the mixture was then filtered. A percent reduction in contaminants of 79% was obtained. A second sample of the mixture was filtered through the same filter at 0.35 bar (0.5 psid). A percent reduction in contaminants of 92.8% was obtained. This further suggests that low temperature processing produces a precipitate that can extrude through even tight filter media, but that can be retained once that filter medium has a significant dirt cake to provide supplemental interception.

### Experiment II-18

Synthetic hard water was prepared by adding CaCl₂ (0.120 g), MgCl₂ (0.126 g), NaHCO₃ (0.201 g), and KCl (0.100 g) to distilled water (500 g). The salts were dissolved in the water within 15 minutes using a magnetic stirrer, to produce a moderately hard and moderately alkaline water. Two samples were created: a mixture of 10 g B test sample and 10 g distilled water (1:1 ratio), and a mixture of 10 g B test sample and 10 g synthetic hard water (1:1 ratio). Acetic acid (0.091 g) was added to each sample. Both samples were heated for 8 minutes to reach 190 degrees F. After filtration, the distilled water sample demonstrated a 93.1% reduction in contaminants, and the synthetic hard water sample demonstrated a non-readable (<69%) reduction in contaminants. The pH of the distilled water and B test sample mixture was 3.8. The pH of the hard water sample and B test sample mixture was 4.0. The pH of the hard water itself was 7.8. This result indicates that hard water can impact the process under these conditions.

### Experiment II-19

Experiment II-18 was repeated using 0.102 g acetic acid in the distilled water sample, and 0.099 g acetic acid in the hard water sample. The sample was also heater faster, to reach 190 degrees F. in 3 minutes. A 93.5% reduction in contaminants was achieved with the hard water sample, and a 94.4% reduction was achieved with the distilled water sample. These conditions appear to eliminate the impact of hard water.

### Experiment II-20

Experiment II-18 was repeated using 0.035 g acetic acid in the hard water sample and 0.030 g acetic acid in the distilled water sample. A 93.2% reduction in contaminants was achieved in the hard water sample, and a 92.4% reduction in contaminants was achieved in the distilled water sample. In this case, the terminal process temperature of 190 degrees F. was reached in 6 minutes. No notable reduction in performance was achieved when using hard water. This indicates that using hard and alkaline water does not negatively affect the process in most cases. Furthermore, in a process using a condenser to recycle the water, any hardness/alkalinity of the water will be eliminated through repeated distillation of the water. This suggests that in a system fitted with a condenser, water pre-treatment may not be necessary.

### Series III

Another sample of TEG was obtained, this time from a different natural gas processing plant, from the “lean” portion of the machine, after the boiler. This unfiltered sample is referred to herein as the D test sample. This sample was filtered through a Whatman #5 filter paper (2.5 micron) and showed an optical density of 20.08 AU as compared to the original unfiltered D test sample which showed an optical density exceeding 30 AU. It was observed that the 110 mm diameter filter paper used to filter the D test sample struggled to operate against the high particulate contamination of this sample, and only small samples of the fluid could be filtered. Based upon these observations, the D test sample is assumed to be heavily loaded with particulate contamination.

### Experiment III-1

The D test sample (10 g) was combined with water (10 g) and acetic acid (0.086 g). This corresponds to a water to glycol ratio of 1:1 and an acid content of 0.86%. The mixture was brought to 190 degrees F. in a 50 mL beaker. The sample was filtered through 1-micron glass filter medium. The optical density of the sample was measured. A percent reduction in contaminants of 94.6% was achieved. This result is indistinguishable from the results obtained with the C test sample.

### Experiment III-2

Experiment III-1 was repeated using 0.080 g acetic acid, and in a stainless steel beaker of about 50 mL volume. A temperature of 185 degrees F. was reached in 7.5 minutes at a reduced heater setting, to adjust for the high thermal conductivity of the beaker. A percent reduction in contaminants of 93.9% was obtained. A residue remained on the stainless steel surface of the beaker, and this was easily flushed with cold water and with agitation. It was observed that approximately 6.5% of the water was lost during heating. This experiment indicates that metallic heating surfaces are suitable in this process.

### Series IV

A fourth series of experiments was conducted, in order to (1) test the use of coagulants for cleaning contaminated glycol, and (2) test various filtration media, including adsorbents, for cleaning contaminated glycol.

Unless otherwise noted, filtration was carried out using 47 mm filter media disks cut with a die stamping tool and mounted into Nalgene filter assemblies. Vacuum stopcock grease was applied to the filter apparatus gaskets.

Unless otherwise noted, filtration was carried out at low pressure of 0.2 bar vacuum. Filtrate was collected in a vacuum flask for polycarboxyl assay per the previously-developed optical assay at 680 nm wavelength.

For treatment with alum as a coagulant, a solution of 1.34 grams aluminum sulfate hydrate (Al₂(SO₄)₃.16H₂O) in 500 g of distilled water was prepared (‘alum solution’). For treatment with a flocculant, a solution of 2.0 g Merquat-100 in 500 g of distilled water was prepared (‘Merquat-100 solution’). Because of the extensive water of hydration, the actual weight of aluminum sulfate in water was significantly reduced. The same applies to the Merquat-100 concentration, which is only 32% solids.

For acetic acid treatment, 1% by weight of glacial acetic acid in distilled water was used.

A sample of TEG was selected that when treated with acetic acid, did not produce a precipitate that could be significantly filtered using conventional micro-glass filter medium. This sample appeared to be contaminated with nearly pure low molecular weight (LMW) poly(carboxyl) compounds with a modest amount of very low molecular weight (VLMW) poly(carboxyl) compounds. This source material is called “Crestwood A”.

### Experiment IV-1

A sample of Crestwood A (20 g) was mixed with 19 g of alum solution and 1 g of Merquat-100 solution. The mixture was stirred and allowed to sit for 15 minutes prior to filtration through a set of micro-porous membranes. This result was compared to filtration through conventional micro-glass filter media having an estimated absolute rating of 5 microns.

The membranes used were a Whatman 1-micron polyethersulfone membrane and a Whatman 0.45 micron cellulose acetate membrane. Applied pressure during filtration was 12 psid. The optical density of the original fluid, and effluent through these various filter media is shown in Table 18.

TABLE 18

| Sample | | |
|---|---|---|
| No. | Filter Media | Optical Density |
| 1 | None | 16.9 |
| | (original Crestwood A Fluid) | (determined by serial dilution) |
| 2 | Whatman 1 micron PES | 0.869 |
| 3 | Whatman 0.45 micron CA | 0.833 |
| 4 | 2 Layers Jonell 1-micron | >3.5 unreadable |
| | microglass | |

Passage of the effluent emerging from microporous membrane filtration through a bed of Fuller's Earth produced a fluid having an optical density of <0.3. This further enhancement in effluent quality was consistent across many tests and may be the result of adsorption of non-precipitated contaminants. Without being limited by theory, it is believed that it is likely that a portion of this material is very low molecular weight poly(carboxyl) compounds that are the precursors to the formation of higher molecular weight poly(carboxyl) compounds. Tests with coconut-shell activated carbon having almost a pure micro-pore structure had no significant impact on effluent quality, which may indicate that the colored impurity is too large to fit within the extremely small pores of this specific activated carbon.

The above results suggest that particulate filtration alone does not provide the best optical clarity to the effluent and that supplemental exposure to a suitable adsorbent can further improve effluent quality. Processing through an adsorbent can be accomplished, for example, using a separate bed of adsorbent, through the use of adsorbent as a filter pre-coat, or as adsorbent-impregnated or loaded filter medium that can combine the microporous structure with a suitable adsorbent. In some cases, layers of filter medium can be laminated together to create a multi-functional filter medium with both micro-porous filtration and adsorbent in a single integrated structure.

### Experiment IV-2

Distilled water (20 g) and 20 g of Crestwood A glycol was mixed, without the application of heat. After 15 minutes, this mixture was filtered through a filter paper having an estimated 0.3 micron absolute rating at 0.2 bar vacuum. An effluent with A.U.>3.5 (unreadable) was obtained. This suggests that pretreatment (either with acetic acid or coagulant) is a requirement and that the filter paper alone cannot accomplish any significant purification of the Crestwood LMW sludge sample.

The above suggests that particulate filtration alone does not provide the best optical clarity to the effluent and that supplemental exposure to a suitable adsorbent will further improve effluent quality.

### Experiment IV-3

Experiment IV-1 was repeated with a sample called “Hanlon”, known to primarily contain HMW polycarboxyl sludge. Alum solution (19 g), Merquat-100 solution (1 g) and Hanlon TEG (20 g) had an initial optical density of 60.23 A.U. This was filtered through a disk of 0.3 micron filter paper, and an effluent with A.U.=0.036 was obtained. This suggests that treatment with alum/poly(DADMAC) will reduce HMW sludge as much as 99.9%. Hence, the described process can clean both LMW (Example 1) and HMW sludge.

### Experiment IV-4

Crestwood A (20 g) was mixed with 20 g of 1% acetic acid in distilled water and heated to 190 degrees Fahrenheit. This was immediately filtered through two layers of 0.3 micron filter medium to assess if the acetic acid treatment works with a sample contaminated with LMW sludge. An effluent with A.U.=0.769 was obtained. This suggests that fine filtration can filter the precipitates emerging from acetic acid treatment.

### Experiment IV-5

Alum solution (19 g) and Merquat-100 solution (1 g) was loaded into a 50-ml beaker. Crestwood A (20 g) was loaded into a separate 50-ml beaker. Both beakers were placed into a refrigerator at 7 degrees Celsius for 90 minutes. A Nalgene filter disk holder was prepared with two layers of 0.3 micron absolute filter paper, and this filter apparatus was placed into the refrigerator to cold soak. After the components and ingredients were cold soaked, the ingredients were mixed, and retained in the refrigerator for 15 minutes. The mixture was then filtered through the cold filter apparatus. It was observed that the use of the 0.2 bar vacuum was not able to force the emergence of an effluent from the filter, and the vacuum was increased to 0.7 bar to obtain a suitable sample. The effluent displayed A.U.=0.669. This was similar to the result obtained during room temperature processing. It is suggested that a reason to heat the ingredients to room temperature is to make filtration possible at lower pressure, but that the precipitation of contaminants will commence immediately even at moderately cold temperatures.

### Experiment IV-6

A large-scale comparative test of the two treatments was carried out against a broad spectrum of dirty glycol samples, obtained from a wide range of processing plants in the states of Texas, Louisiana, and Oklahoma. In each case, a sample of dirty glycol was treated with hot acetic acid or room-temperature alum/poly(DADMAC) and then filtered through a single layer of 0.3 micron absolute filter paper. All samples were filtered at a fixed 0.2 bar vacuum.

Sample number 5 is assessed as being composed of nearly pure HMW sludge components, while the other samples were contaminated with some mixture of HMW and LMW poly(carboxyl) sludge. Results are shown in Table 19.

TABLE 19

| | Optical Density | | |
|---|---|---|---|
| | | | Treated with |
| Sample | | Treated with | Alum/ |
| No. | Untreated | Acetic Acid | poly(DADMAC) |
| 1 | 18.08 | 0.338 | 0.281 |
| 2 | 10.19 | 0.229 | 0.218 |
| 3 | 2.504 | 0.164 | 0.233 |
| 4 | 1.118 | 0.062 | 0.147 |
| 5 | 20.17 | 0.094 | 0.105 |
| 6 | 2.233 | 1.350 | 1.058 |

While the above description provides examples of one or more processes or apparatuses, it will be appreciated that other processes or apparatuses may be within the scope of the accompanying claims.

1. A process for cleaning contaminated fluid, the process comprising:
a) obtaining a contaminated fluid that has been contaminated with poly(carboxyl) compounds;
b) combining the contaminated fluid with an acid and water to yield a mixture in which at least some contaminants of the contaminated fluid are insoluble, the insoluble contaminants precipitating to yield precipitated contaminants;
c) separating at least a portion of the precipitated contaminants from the mixture; and
d) separating at least a portion of the acid and at least a portion of the water in the mixture from the fluid in the mixture to yield cleaned fluid.

2. The process of claim 1, wherein in step b) the temperature of the mixture is elevated.

3. The process of claim 2, wherein the temperature is elevated to at least about 140 degrees F.

4. (canceled)

5. (canceled)

6. (canceled)

7. The process of claim 1, wherein step c) is carried out at most 20 minutes after the initiation of step b).

8. The process of claim 1, wherein the contaminated fluid is not filtered prior to step b).

9. The process of claim 1, wherein the contaminated fluid comprises at least one of triethylene glycol, diethylene glycol, tetraethylene glycol, and ethylene glycol.

10. (canceled)

11. The process of claim 1, wherein the acid comprises at least one of acetic acid, sulphuric acid phosphoric acid, and hydrochloric acid.

12. The process of claim 1, wherein the acid comprises acetic acid.

13. (canceled)

14. The process of claim 1, wherein the water to contaminated fluid ratio in the mixture is between about 1:2 and about 2:1 by weight.

15. (canceled)

16. (canceled)

17. The process of claim 1, wherein the amount of acid in the mixture, as measured by weight % of the contaminated fluid in the mixture, is between about 0.25% and about 30%.

18. (canceled)

19. (canceled)

20. (canceled)

21. The process of claim 1, wherein step c) comprises at least one of:
i) initially filtering the mixture through a filter paper with an average pore size of 1.0 to 2.5 microns at a pressure less than 0.5 bar by operating at a low flow rate through the filter medium or by restricting applied force across the filter medium; and
ii) filtering the mixture through a sub-micron filter media at a pressure of less than 0.3 bar by operating at a low flow rate through the filter medium or by restricting applied force across the filter medium.

22. (canceled)

23. The process of claim 1, wherein step c) comprises at least one of filtering the mixture through sub-micron filter media and filtering the mixture through an adsorbent.

24. (canceled)

25. The process of claim 1, further comprising:
e) after step c), contacting the mixture with an ion exchange resin to remove salts from the mixture, wherein the ion exchange resin comprises at least one of a cationic exchange resin and a mixed cationic and anionic exchange resin.

26. (canceled)

27. (canceled)

28. (canceled)

29. (canceled)

30. The process of claim 1, wherein the contaminants comprise at least one of high molecular weight poly(carboxyl) compounds and low molecular weight poly(carboxyl) compounds.

31. (canceled)

32. The process of claim 1, wherein step d) comprises evaporating the portion of the water and the portion of the acid from the mixture, and condensing the evaporated water and acid and recycling the condensed water and acid to step b).

33. (canceled)

34. (canceled)

35. (canceled)

36. The process of claim 1, wherein the contaminated fluid comprises at least one of lube oil and glycol.

37. A process for precipitating contaminants from contaminated fluid, the process comprising:
a) combining the contaminated fluid with water and acid to yield a mixture in which at least a portion of the contaminants are insoluble, the insoluble contaminants precipitating to yield precipitated contaminants.

38. The process of claim 37, wherein step a) is carried out at an elevated temperature.

39. The process of claim 38, wherein the elevated temperature is at least about 140 degrees F.

40. (canceled)

41. (canceled)

42. (canceled)

43. The process of claim 37, wherein the contaminated fluid is not filtered prior to step a).

44. The process of claim 37, wherein the contaminated fluid comprises at least one of triethylene glycol, diethylene glycol, tetraethylene glycol, and ethylene glycol.

45. (canceled)

46. The process of claim 37, wherein the acid comprises at least one of acetic acid, sulphuric acid, phosphoric acid, and hydrochloric acid.

47. The process of claim 37, wherein the acid comprises acetic acid.

48. (canceled)

49. The process of claim 37, wherein the water to fluid ratio in the mixture is between about 1:1 and about 1:2 by weight.

50. (canceled)

51. (canceled)

52. The process of claim 37, wherein the amount of acid in the mixture, as measured by weight % of the fluid in the mixture, is between about 0.25% and about 30%.

53. (canceled)

54. The process of claim 37, wherein the contaminants comprise at least one of high molecular poly(carboxyl) compounds and low molecular weight poly(carboxyl) compounds.

55. (canceled)

56. (canceled)

57. A process for cleaning contaminated triethylene glycol, the process comprising:
a) combining the contaminated triethylene glycol with acetic acid and water, wherein the water to glycol ratio is between about 2:1 and about 1:2 by weight and the amount of acetic acid in the mixture, as measured by weight % of the glycol in the mixture, is between about 0.25% and about 30%, wherein at least some high molecular weight polar contaminants in the glycol are insoluble in the resulting mixture, and wherein the insoluble high molecular weight polar contaminants precipitate from the mixture to yield precipitated high molecular weight polar contaminants;
b) separating at least a portion of the precipitated high molecular weight polar contaminants from the mixture; and
c) separating at least a portion of the water and acetic acid in the mixture from the triethylene glycol in the mixture by evaporating the portion of the water and acetic acid.

58. (canceled)

59. The process of claim 57 wherein the mixture of water, acid and glycol is heated to greater than 140 degrees F.

60. The process of claim 57, wherein step b) comprises at least one of filtering the mixture through sub-micron filter media and filtering the mixture through an adsorbent.

61-66. (canceled)
